(19) United States
(12) Patent Application Publication
(10) Pub. No.: US 2003/0224349 A1
(43) Pub. Date: Dec. 4, 2003

(54) Rapid assay to assess the potential for glucocorticoid analogs to promote the differentiation of human osteoblasts and predict bone safety

(75) Inventor: Leonard Buckbinder, Pawcatuck, CT (US)

Correspondence Address: PFIZER INC, 150 EAST 42ND STREET, 5TH FLOOR - STOP 49, NEW YORK, NY, 10017-5612, US

(73) Assignee: Pfizer Inc.

(21) Appl. No.: 10/452,853

(22) Filed: Jun. 2, 2003

Related U.S. Application Data
(60) Provisional application No. 60/385,385, filed on May 31, 2002.

(51) Int. Cl. C12Q 1/00; C12Q 1/42

(52) U.S. Cl. 435/4; 435/21

(57) ABSTRACT

This invention relates to in vitro screening and assay methods for the selection of anti-inflammatory compounds that exhibit minimized effects on the differentiation of human osteoblasts.

### FIELD OF THE INVENTION

This invention relates to in vitro screening and assay methods for the selection of anti-inflammatory compounds that exhibit minimized effects on the differentiation of human osteoblasts.

### BACKGROUND

The adrenal cortex produces and secretes sex steroids and corticosteroids. Corticosteroids have been in pharmaceutical use for over 40 years and have become indispensable in controlling a number of different disease states. Corticosteroids are categorized as glucocorticoid or mineralcorticoid as determined by their effects, with an overlap of effects being common.

The principal human glucocorticoid is hydrocortisone. Glucocorticoids used in therapy are synthetic preparations that mediate many disease states. They may be administered in physiologic or supra-physiologic doses for their anti-inflammatory effects in diseases such as asthma or arthritis or for their immunosuppressive effects.

Glucocorticoids are used to treat a wide variety of inflammatory diseases including asthma, allergy, and lupus and are established disease-modifying drugs used for the treatment of rheumatoid arthritis. However, many serious side effects limit their chronic use and a long-standing medical goal has been the identification of glucocorticoid analogs (compounds that bind and mediate their activity through the glucocorticoid receptor) with improved safety (Buckbinder, L. and Robinson, P. C.: The Glucocorticoid Receptor: Molecular Mechanisms and New Therapeutic Opportunities, Current Drug Targets—Inflammation and Allergy 1:127-136, 2002).

Glucocorticoids bind to the glucocorticoid receptor in the cytoplasm. The hormone-receptor complex is then translocated to the nucleus where it binds and modulates the activity of a number of diverse genes. It appears that all cells have glucocorticoid receptors and therefore are responsive to the presence of the glucocorticoid.

One of the most common side effects of the administration of supraphysiologic doses of glucocorticoids is induced osteoporosis. Glucocorticoids suppress intestinal calcium absorbtion, decrease sex hormone production, and inhibit bone formation. Such side effects are most common in patients who are postmenopausal, have restricted mobility or are over 50 years old.

Bone formation involves three steps: production of the extracellular organic matrix, mineralization of the matrix to form bone and bone remodeling by resorption and reformation. Osteoblasts, derived from mesenchymal stem cells of the bone marrow stroma, are required in the bone formation process. Osteoblasts synthesize precursors of collagen I which comprises over 90% of the organic matrix of bone and in addition to producing osteocalcin and proteoglycans rich in alkaline phosphatase.

The osteoblast is a major target for the bone side effects (Manolagas S C, Weinstein R S: New developments in the pathogenesis and treatment of steroid-induced osteoporosis. J Bone Miner Res 14:1061-1066, 1999), which include osteoporosis, osteopenia, and bone fracture.

In glucocorticoid treated patients and animals, osteoblast numbers and activity are reduced and there is evidence of increased apopotosis (Weinstein R S, Jilka R L, Parfitt A M, Manolagas S C: Inhibition of osteoblastogenesis and promotion of apoptosis of osteoblasts and osteocytes by glucocorticoids. Potential mechanisms of their deleterious effects on bone. J Clin Invest 102:274-282, 1998; Weinstein R S, Nicholas R W, Manolagas S C: Apoptosis of osteocytes in glucocorticoid-induced osteonecrosis of the hip. J Clin Endocrinol Metab 85:2907-2912, 2000).

Glucocorticoids inhibit osteoblast proliferation and stimulate their terminal differentiation to bone nodules. This is true for both the immature, alkaline phosphatase negative progenitor cells and for the more differentiated, alkaline phosphatase expressing cells (Turksen K, Aubin J E: Positive and negative immunoselection for enrichment of two classes of osteoprogenitor cells. J Cell Biol 114:373-384, 1991).

It would be desirable to identify glucocorticoids or glucocorticoid analogs that exhibit decreased effect on osteoblast differentiation.

It is therefore an object of this invention to develop a technique for distinguishing the osteoblast differentiation effect of compounds that bind to the glucocorticoid receptor and which exhibit anti-inflammatory effectiveness.

It is an object of this invention to develop a test that provides results in a shorter time period that the 28 days required for current tests.

It is another object of this invention to provide an assay to determine the dose dependency of osteoblast differentiation on the quantity of a test compound present.

### SUMMARY

We have developed a novel method to screen for differentiation of osteoblasts by measuring the activity of alkaline phosphatase. The advantages of this method are that 1) it saves time (<13 days vs. 28 days for current assays), 2) it provides cost savings from reduced human time, reagents and compound, and 3) results are easily quantitated (spectrophotometer reading vs qualitative micrographs in prior art methods). Anti-inflammatory glucocorticoid analogs that do not promote osteoblast differentiation are expected to show reduced bone side effects.

A method for studying the effects of glucocorticoid on the differentiation of primary cultures of rodent osteoblasts is well known. (Bellows C G, Aubin J E, Heersche J N: Physiological concentrations of glucocorticoids stimulate formation of bone nodules from isolated rat calvaria cells in vitro. Endocrinology 121:1985-1992, 1987; Bellows C G, Aubin J E, Heersche J N, Antosz M E: Mineralized bone nodules formed in vitro from enzymatically released rat calvaria cell populations. Calcif Tissue Int 38:143-154, 1986). However, the development of a system for assaying the effect of glucocorticoids on primary human osteoblasts has proven to be more challenging. It has now been determined that human osteoblasts from young donors can be utilized in an in vitro system to determine the effect of glucocorticoids on primary human osteoblasts.

After a course of 28 days of glucocorticoid treatment, nodule formation and mineral deposition in confluent cultures of osteoblasts were assessed by micrographic analysis using Von Kossa or alkaline phosphatase staining. Cells treated with glucocorticoid showed marked dose-dependent increase in mineral deposition as compared to vehicle control. The method was validated with a novel, non-steroid glucocorticoid receptor ligand that showed anti-inflammatory efficacy, inhibiting the expression of inflammatory mediators (MMP13 and IL-8) in a human cell-based assay. Analysis of this proprietary compound (DAGR1) showed that it did not promote mineralization and caused the production of much less alkaline phosphatase than the dexamethasone standard.

To facilitate assessment of GCs and new analogs, a novel 96-well colorimetric assay was developed to detect alkaline phosphatase activity in treated cultures after only 7 to 10 days of treatment. The alkaline phosphatase liquid assay results correlated with the Von Kossa staining at the longer 28-day time point. This facile method is predictive for compounds that promote osteoblast differentiation.

Utilizing either of the nodule or liquid alkaline phosphatase assays, glucocorticoid analogs encompassing steroid and non-steroidal compounds having anti-inflammatory activity, and whose action is mediated by binding to the glucocorticoid receptor, can be screened to determine which have reduced capacity to differentiate human osteoblasts. Thus the human nodule and alkaline phosphatase assays are useful for selecting glucocorticoid receptor-based anti-inflammatory drug candidates having improved bone safety.

### BRIEF DESCRIPTION OF THE DRAWINGS

FIG. 1 is a table (identified as Table 1) showing the Kd for various glucocorticoids.

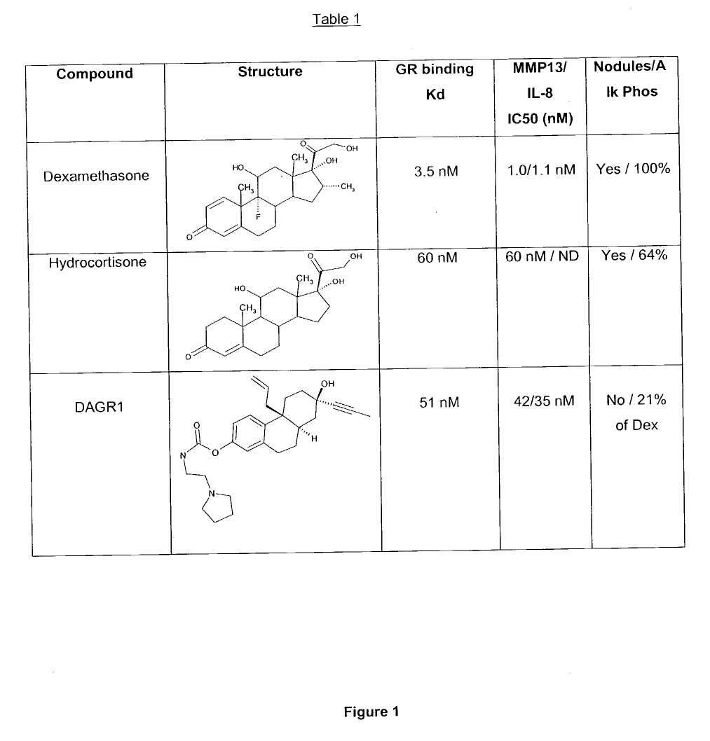

FIG. 2 is a chart showing the dose-dependent terminal differentiation of human osteoblasts by dexamethasone, hydrocortisone, and DAGR1.

FIG. 3 shows a series of nodule micrographs highlighted by von Kossa staining and demonstrating the varying degree of alkaline phosphatase production in osteoblasts utilizing different glucocorticoids.

### DETAILED DESCRIPTION OF THE INVENTION

To determine the applicability of the assay to compounds that bind to the glucocorticoid receptor, candidates for testing were screened to determine whether they bind to the glucocorticoid receptor using a competition binding assay. Compounds which bind the glucocorticoid receptor (Kd<10 uM) as determined by competition binding using a known reference compound tracer are selected as candidates for stage two testing.

Such competition binding assays are well known in the art and can utilize liver extracts or recombinant protein as a source of glucocorticoid receptor and a radioactive ligand such as dexamethasone (New England Nuclear, Boston, Mass.) or a flourescent ligand such as Fluormone (Panvera, Madison, Wis.). Dexamethasone is a potent synthetic glucocorticoid while hydrocortisone is the naturally occurring adrenal hormone. Both have the properties of a full agonist and are used clinically. As recorded in Table 1, the Kd measured for dexamethasone is 3.5 nM, hydrocortisone 60 nM, and for DAGR1 52 nM.

Glucocorticoids have anti-inflammatory efficacy in a variety of cell models. Clinically, glucocorticoids set the benchmark for anti-inflammatory therapeutics. The anti-inflammatory properties of dexamethasone, hydrocortisone, and DAGR1 were shown utilizing a model chondrocyte cell line, SW1353, a cell type relevant to the cartilage damage observed in rheumatoid arthritis.

For efficacy testing, 10,000 SW1353 cells (ATCC# HTB-94) were plated in 96 well plates in DMEM with 10% Fetal Bovine Serum (FBS). The next day media was changed to DMEM without serum.

The following day the cells were treated with the reference compounds dexamethasone and hydrocortisone and the test compound in a range of doses from 1 nM to 10 uM along with the pro-inflammatory stimulus, 10 ng/ml IL-1β (R&D Systems). After 24 hours the amount of MMP13 and IL-8 released into the media was quantified using commercial ELISA assays (Amersham and R&D Systems, respectively).

The concentration of compound reducing the release of MMP13 or IL-8 50% (IC50) was determined from the plot of the drug dose vs. percent inhibition relative to the vehicle control. Table 1 shows the results of such an experiment. Dexamethasone demonstrates potent inhibition of both IL-8 and MMP-13 induction by IL-1β with an IC50 of about 1 nM. Cortisol has an anti-inflammatory potency of 60 nM in MMP13. DAGR1's anti-inflammatory potency is greater than that of hydrocortisone, but less than that of dexamethasone (42 nM in MMP13 and 35 nM in IL-8). This shows that in vitro, DAGR1 has anti-inflammatory potency of clinically used drugs.

Candidates which demonstrate anti-inflamatory activity in the first phase of testing are then subjected to the alkaline phosphatase assay. For alkaline phosphatase testing, primary human osteoblasts (Biowhittaker CC-2538) were plated at a density of 4,400 cells per well in 24-well plates containing OGM medium (Biowhittaker CC-3207) with 10% Fetal Bovine Serum (FBS). The next day media was changed to OGM medium supplemented with 0.1 mM ascorbic acid and 10 mM □-glycerophosphate (differentiation supplements, Biowhittaker CC-4194), and treated with reference and test compounds in a range of doses from 100 nM to 10 uM). Fresh media was added 2× per week.

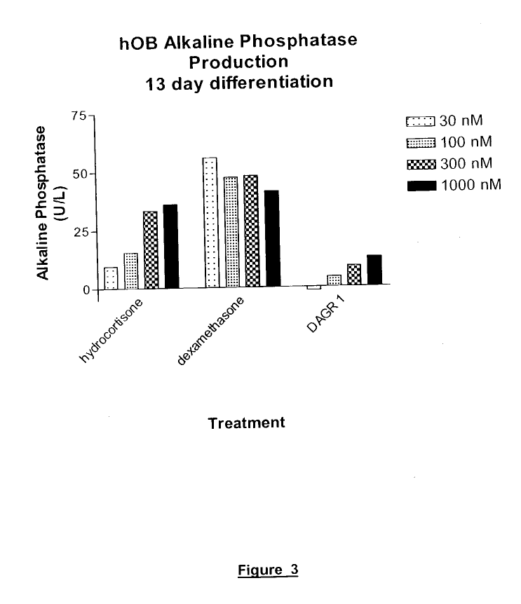

After 13 days the cells were harvested and extract prepared for analysis. FIG. 3 shows nodule micrographs obtained utilizing von Kossa staining. These micrographs show that DAGR1 is differentiated from glucocorticoids by reduced production of alkaline phosphatase in osteoblasts. Alkaline phosphatase production, a classical marker of osteoblast differentiation, is stimulated by hydrocortisone and dexamethasone but remains at minimal levels with DAGR compound treatment (33% and 21% of hydrocortisone or dexamethasone maximum). Similar were obtained when cells were analyzed at 7 and 10 days (data not shown).

Differentiation supplement medium was changed every three to four days with fresh test compound addition each time. After seven to thirteen days, the amount of alkaline phosphatase produced by the cultures was quantified using commercial alkaline phosphatase reagent (ALP Reagent) and standard (Sigma M2266 and 245-10, respectively).

Cells were rinsed with 0.9% NaCl, and lysed in alkaline phosphatase assay buffer (150 mM Tris-HCl pH 8, 0.1 mM MgCl₂, 0.1 mM ZnCl₂, 1% Triton X-100). Prewarmed ALP Reagent was added to lysate or to serial dilutions of the alkaline phosphatase standard and incubated at 37° C. for 30 minutes.

The calorimetric endpoint of absorbance at 405 nm was read on a SpectraMax 340 microplate reader (Molecular Devices), and after extrapolation from standard curve, values were reported in units of enzyme activity per volume.

Analysis of alkaline phosphatase activity produced by human osteoblasts treated for 13 days with different compounds is shown in FIG. 3. Even the lowest concentrations of dexamethasone (30 nM) leads to maximum alkaline phosphatase expression in treated cultures, (a dose response with lower dexamethasone concentrations demonstrates that the induction is dose-dependent, data not shown). Hydrocortisone stimulates a dose dependent increase in alkaline phosphatase expression, reaching about 64% of the dexamethasone maximum at 1 uM. DAGR1 shows weak induction of alkaline phosphatase activity. Although DAGR1's anti-inflammatory activity is more potent than hydrocortisone (42 vs 60 nM IC50), its ability to potentiate alkaline phosphatase expression is only about 33% of hydrocortisone.

As an independent confirmation of the results obtained by the alkaline phosphatase test and an alternative measure that could be used for screening, the compounds were subjected to the nodule assay. Primary human osteoblasts were cultured as for the alkaline phosphatase assay. After 28 days of test compound treatment, the cultures were fixed with 70% ethanol for 10 minutes, stained with 5% silver nitrate solution and exposed to bright light (Polaroid imaging system with 4×150 watt lamps) for 20 to 30 minutes. Wells were rinsed with water, air dried and nodules documented by micrographs at 300× magnification.

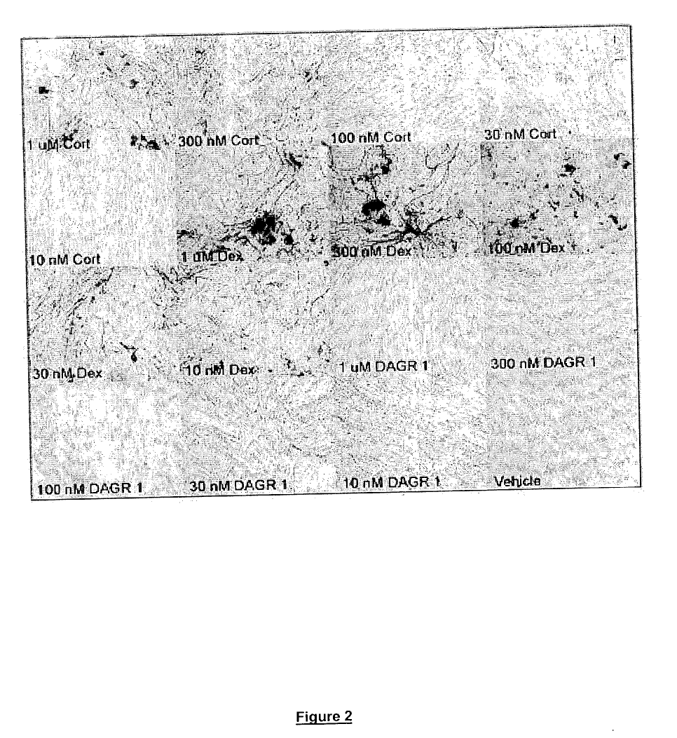

The micrographs of FIG. 2 show opaque regions of brown or black staining, indicating mineral deposition or nodule formation. Both dexamethasone and hydrocortisone show dose-dependant nodule formation while similar concentrations of the DAGR1 test compound have little effect on the differentiation of human osteoblasts. The Von Kossa staining confirms the results of the alkaline phosphatase assay, that dexamethasone and hydrocortisone have show a dose dependent promotion of osteoblast differentiation, while DAGR1 is nearly free of this activity. In other words, at concentrations producing equal anti-inflammatory efficacy, DAGR1 shows greatly reduced osteoblast differentiation activity compared to clinical standards. Thus, this method provides a screening strategy, using either Von Kossa or alkaline phosphatase, to identify efficacious glucocorticoid analogs having the potential for improved bone safety.

1. A method of selecting anti-inflammatory compounds that minimize osteoblast differentiation from those which cause higher levels of osteoblast differentiation, comprising:
a) identifying a compound which binds the glucocorticoid receptor;
b) demonstrating anti-inflammatory activity of the compound; and
c) determining the capacity of the compound to stimulate the differentiation of osteoblasts.

2. A method to identify agonists of the glucocorticoid receptor that dissociate anti-inflammatory efficacy from bone side effects, comprising:
a) identifying a compound which binds the glucocorticoid receptor (Kd<10 uM) as determined by competition binding using a known reference compound tracer;
b) demonstrating anti-inflammatory activity of the compound utilizing one or more cell-based assays and/or animal models; and
c) determining the capacity of the compound to stimulate the differentiation of osteoblasts.

3. The method of claim 2 where the differentiation of osteoblasts is determined by the alkaline phosphatase method.

4. The method of claim 2 where the differentiation of osteoblasts is determined by Von Kossa staining.

5. The assay of claim 3 where alkaline phosphatase activity is determined by colorimetric methods.

6. The method of claim 2 where the anti-inflammatory activity is demonstrated in a cell-based assay.

7. The method of claim 6 where the cell-based assay utilizes SW1353 cells.

8. An assay to determine the effect on osteoblast differentiation of an anti-inflammatory compound comprising cultivating primary human osteoblasts in the presence of the compound and determining the amount of osteoblast differentiation of the cells after a period of from about 7 to about 13 days.

9. The method of claim 8 where the detection of differentiation is determined by alkaline phosphatase activity.

10. An assay to determine the dose dependent effect on osteoblast differentiation of an anti-inflammatory compound comprising
a] cultivating primary human osteoblasts in the presence of a first amount of the compound;
b] cultivating primary human osteoblasts in the presence of a different amount the compound;
c] repeating step b with as many different amounts of the test compound as desired; and
d] determining the amount of osteoblast differentiation at each concentration of added component after a period of from about 7 to about 13 days.
